(19) United States
(12) Patent Application Publication
(10) Pub. No.: US 2011/0106142 A1
(43) Pub. Date: May 5, 2011

(54) SUTURING INSTRUMENTS AND SUTURING SYSTEMS

(75) Inventors: Wouter Van Furth, Hilversum (NL); Marc Padberg, Enschede (NL); Luc De Vries, Diepenheim (NL); Mars Holwerda, Arnhem (NL); Eric Windeler, Toronto (CA); Libbe Jonkman, Drachten (NL); Ingeborg Griffioen, Deventer (NL); Berry Meems, Luttenberg (NL); Iris Hobo, Olst (NL)

(73) Assignee: SUTUREAID HOLDINGS B.V., Ede (NL)

(21) Appl. No.: 12/863,969

(22) PCT Filed: Jan. 23, 2009
(86) PCT No.: PCT/EP2009/050811

§ 371 Date: Nov. 30, 2010

(30) Foreign Application Priority Data
Jan. 24, 2008 (US) 61023401

(51) Int. Cl. A61B 17/04; A61B 17/06

(52) U.S. Cl. 606/205; 206/438

(57) ABSTRACT

A needle storage and disposal cartridge is loaded with a needle receptor, and or suture and needle. By insertion of a forceps arm into the cartridge the needle receptor can be affixed to an arm of the forceps, and similarly removed after use. The forceps may conveniently be used for gripping and retaining a needle during suturing. A number of alternative suturing forceps and needle receptors are disclosed.

### CROSS REFERENCE TO RELATED APPLICATIONS

The present application is a national stage of International Patent Application No. PCT/EP2009/050811, filed Jan. 23, 2009, which claims the benefit of U.S. Provisional Application No. 61/023,401 filed Jan. 24, 2008, and International Application No. PCT/EP2008/053584, filed Mar. 26, 2008 (now abandoned), each of which is incorporated herein by reference in its entirety.

### BACKGROUND OF THE INVENTION

1. Field of the Invention

The invention relates to surgical instruments and surgical systems to be used for suturing tissue while reducing the possibility of needle perforation accidents before, during and after the suturing process.

2. Description of the Related Art

Standard suturing instruments and techniques present significant risks to both patient and surgeon by way of possible glove perforation accidents in which a suture needle penetrates the surgeon's glove. Such needle stick accidents may allow pathogenic organisms such as, but not limited to, the hepatitis virus B, the hepatitis virus C and the human immunodeficiency virus (HIV) to be transmitted from the patient to the practitioner.

Conversely, a perforation accident may cause a break in the sterile barrier between practitioner and patient, which increases the risk of the patient's wound becoming infected. In the context of surgery, accidental piercing of part of the patient's organism during suture may be referred to as a perforation incident. In the following, reference to needle-stick and perforation accidents will be used interchangeably.

One approach to help avoid this problem involves the use of surgical forceps of the kind described in U.S. Patent Application US 2003/0045833 A1. The surgical forceps described in that application has near the distal end at the outside of an arm of the forceps a flexible material that can be used for manipulating a surgical needle during suturing in order to attempt to prevent needle perforation accidents.

Among the drawbacks of these and other surgical forceps is that when suturing using a surgical needle and a suture attached thereto, the tissue may sustain damage. This is a particular liability where delicate tissue is concerned through which it is difficult to pass the surgical needle without causing tissue damage. The point of the surgical needle initially pushes the tissue forward to subsequently lance it, which causes damage to the tissue. Moreover, in the surgical forceps described in the above application, the placement of the flexible material on the outside of the arm at a location away from the distal end of the arm requires release of the tissue in order to receive the needle. Alternately, the instrument may be pushed further into the wound thereby increasing the likelihood of damaging the tissue and/or previously tied sutures.

Other approaches to this problem are described in WO 2005/094698 (PCT/EP2005/003476), U.S. patent application Ser. No. 11/025,727 and U.S. patent application Ser. No. 10/599,503, which are incorporated herein by reference in their entirety.

Another scenario in which needle perforation accidents may occur is during minimally invasive surgery when the exposed sharp tip of the needle may inadvertently puncture or cut tissue while in the patient.

Thus, there remains a need for suturing forceps, and suturing systems, which minimize the risk of glove perforation accidents and other types of needle perforation accidents and preferably tissue damage before, during and after the suturing procedure.

### BRIEF SUMMARY OF THE INVENTION

The invention is directed to surgical instruments and surgical systems with which needle perforation accidents can be avoided, tissue damage during suturing can be reduced and which allows suturing to be performed more easily.

The following description is made by way of example and is not intended to limit the invention as set out in the appended claims.

For the purposes of this application the term “needle receptor”, “NR” or “bullet” refers to a surgical needle-receiving and affixing portion of the suturing instruments of the present invention. In preferred embodiments the needle receptor (NR) is disposed at a distal end of the suturing forceps with variation from that configuration described herein.

In certain embodiments, the present invention provides a forceps comprising at least a first and second arm that are connected at a proximal end and which may be biased, for example, by a spring means, in an open position and which defines a space between them, which can be reduced or increased and further comprising a needle receiving and affixing NR, in combination with a cartridge for storing and/or disposing of the NR. The NR is preferably positioned at the distal end of a forceps arm. The term “open” in the context of the present invention refers to the position wherein the distal ends of the two arms are apart. The term “closed” refers to the position wherein the distal ends of the two arms are in close proximity or touching.

In certain embodiments, the NR may be removable from the suturing instrument in which case the NR may be disposable. In this context, it will be understood that reference to the NR can also include a mounting portion allowing connection of the NR to a surgical instrument such as a forceps or needle holder.

In certain embodiments, the NR is placed at the distal end of an arm of a forceps at an outside of the end of said arm. In other embodiments, the NR is placed at the distal end of an arm of a forceps at an inside of the end of said arm. In other embodiments, the NR is placed at the distal end of an arm of a forceps at either side of the end of said arm. In yet another embodiment, the NR is integrated at the distal end of an arm of a forceps. In yet another embodiment, the NR may be placed at the distal end of an arm at an inside, outside or side of the end of an arm and extends beyond the distal end of the arm.

In other embodiments, the NR can form part of the tissue-gripping surface of the suturing instrument. In these embodiments, the NR is positioned such that the NR simultaneously supports the tissue to be pierced with a surgical needle and is capable of receiving and affixing the needle thereafter so that the needle can be manipulated with the instrument. This allows the surgical needle to be manipulated safely during suturing without touching the needle with the hands, thereby reducing the possibility of needle perforation accidents. In these embodiments, the NR is preferably positioned at the distal end of an arm of a forceps at an inside and/or side of the end of said arm, but may be placed at any position at the distal end of a forceps arm that allows the NR to simultaneously grip the tissue and receive and affix a surgical needle.

In other embodiments, the NR does not form part of the tissue-gripping surface of the suturing instrument; however, the NR is positioned such that the NR is capable of receiving and affixing the needle while forceps arms grip the tissue. In these embodiments, the NR may be located at the distal end of an arm of a forceps at an outside of the end of said arm.

When suturing tissue using a surgical needle, the forceps and suturing system of the present invention makes it possible to control the suturing process in such a manner that immediately after the point of the surgical needle has pierced the tissue, it is able to pass into the NR where it is retained until removal by the surgeon, first for additional suturing steps, and until suturing is complete, and then for safe and efficient disposal of the sharp needle and NR, preferably with the use of a needle storage and disposal cartridge. This allows the surgical needle to be manipulated safely during suturing without touching the needle with the hands, it allows the forceps and needle to be passed between users safely with the needle tip protected, and it allows for safe and easy disposal of the NR and needle thereby reducing the possibility of needle perforation accidents throughout all steps of the suturing process. It also helps prevent needle perforation accidents that may occur during manipulation of the needle in a patient during minimally invasive surgery, and upon removal of the needle from the patient. The term “needle-affixing material” as referred to herein means a material capable of receiving and removably affixing or retaining a surgical needle. For example, the needle-affixing material may be, without limitation, a soft plastic or a synthetic rubber material.

### BRIEF DESCRIPTION OF THE DRAWINGS

The invention will now be further elucidated by way of exemplary embodiments that form no limitation to the appended claims, and with reference to the following drawings in which:

FIG. 1 shows a needle storage and disposal cartridge loaded with an NR (or bullet) (left panel) and insertion of a forceps arm into the cartridge (right panel);

FIG. 2 shows a suture system component comprising needle storage and disposal cartridges, a mounting or affixing mechanism, for example, a rail for storing the cartridges, NRs, a needle holder and a suturing forceps;

FIG. 3 shows successive stages of loading a needle storage and disposal cartridge with an NR;

FIG. 4 shows successive stages of transferring a NR in a needle storage and disposal cartridge to the distal end of a forceps arm (top panel) and a forceps arm inserted into a chamber NR embodiment to which a needle optionally with a suture thread is attached (bottom panel);

FIG. 5 shows a cartridge suitable for both release of a NR and disposal of a NR with needle and optionally suture thread;

FIG. 6 shows a chamber NR embodiment, gripping an inserted needle for safe storage and needle passing while the needle tip is fully protected;

FIG. 7 shows the sandwich NR embodiment viewed from the bottom (upper left panel) the top (upper right panel) and the side (bottom panel, attached to a forceps arm);

FIG. 8 shows a diagram of the sandwich NR embodiment viewed from the side and attached to a forceps arm and with a needle inserted into a hole at the top of the NR;

FIG. 9 shows the NR-ridge forceps according to the invention;

FIG. 10 shows the NR-ridge forceps in successive stages of receiving and affixing a surgical needle;

FIG. 11 shows several further embodiments of the NR-ridge forceps

FIG. 12 shows an illustration of the fork-tip forceps according to the invention;

FIG. 13 shows another forceps embodiment of the invention;

FIGS. 14A and 14B show diagrams illustrating several further embodiments of the forceps of the present invention; and

FIG. 15 shows diagrams illustrating several further embodiments of the NR ridge.

FIG. 16 shows diagrams illustrating a ‘micro’ forceps suitable for cardiovascular surgery, comprising a relatively small NR provided on a detachable holder.

### DESCRIPTION OF ILLUSTRATIVE EMBODIMENTS

In one aspect, the present invention provides a cartridge for the safe storage and disposal of needles and NR. The cartridge may contain a NR and might also contain a needle or a needle with suture. The NR may be any NR embodiment described herein. The cartridge can be mounted, for example, on a rail or other suitable mounting surfaces in the operating room. The cartridge comprises a housing having an opening through which the user can insert a forceps. Once inserted into the cartridge, the NR is attached to the forceps. In one embodiment, rotation of the forceps inside the cartridge by the user causes the NR to become attached to the forceps. When the forceps is pulled out, the NR is correctly positioned onto the forceps and the sharp tip of the needle is inserted in the NR, enabling a safe transfer of the forceps from, for example, the nurse to the surgeon. Preferably, the NR can only be removed from the cartridge on insertion of and connection to, the correct forceps.

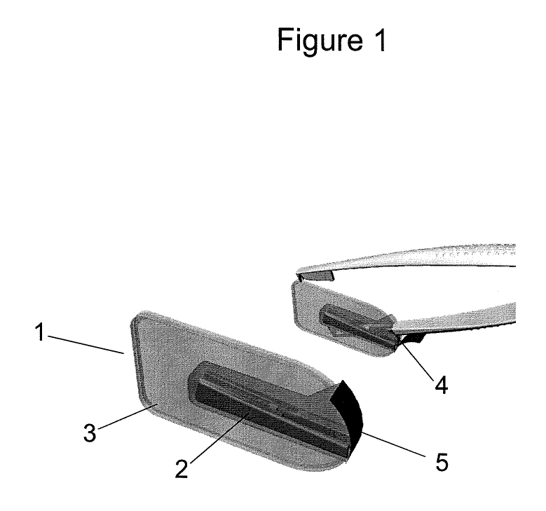

After usage of the NR, the forceps with contaminated NR suture thread is inserted into the cartridge. By rotating the forceps the NR is detached from the forceps and the NR and contaminated suture remain in the cartridge. Preferably, the cartridge comprises a means for indicating that the cartridge is ‘loaded’ to the users, for example with a colored indicator or because it's housing is transparent. Examples of needle storage and disposal cartridges are illustrated in FIGS. 1-5. The housing is preferably a rigid housing, such as hard plastic, that substantially surrounds the NR on all sides. In this manner, access to the NR and to the needle and suture within the housing is precluded until these have been removed through the opening in the correct manner. It will thus be understood that in the preferred embodiment, insertion of a user's fingers through the opening into the interior of the cartridge is precluded. Access to the interior of the cartridge housing can also be prevented by the presence of the NR or its mounting portion.

In one embodiment, a cartridge is provided that is designed to contain two or more NRs. Such cartridges are particularly advantageous in combination with double-armed sutures (i.e., a needle is located at both ends of the suture) and/or in combination with relatively small needles. Cardiovascular sutures, for example, are generally double-armed and the needle attached at each end of the suture is relatively small. A relatively small needle may be used in combination with a relatively small NR, such that the cartridge may be designed to hold multiple of these relatively small NRs.

The cartridge and the NR(s) may be molded together as one piece. For example, a cartridge comprising a housing of hard plastic may be molded together at once with the NR(s). The NR may comprise a soft needle-affixing material such a soft plastic which may be partially covered by a hard plastic supporting material. The NR should easily separate from the cartridge once attached to a forceps arm and should remain attached to the forceps arm when the user pulls the forceps out of the cartridge. Alternatively, the cartridge may be manufactured separately from the NR.

In various embodiments of the invention, the cartridge is used in combination with a forceps comprising a first arm and a second arm connected at a proximal end and defining a space between said first arm and said second arm which can be increased or decreased, said forceps further comprising any of the needle receptor (NR) embodiments described in U.S. application Ser. Nos. 11/025,727 and 10/599,503. In such embodiments, the NR affixes at the distal end of a forceps arm at an inside, outside and/or side of the end of an arm.

In certain embodiments, the cartridge is used in combination with a forceps comprising an NR which is capable of being pierced with a surgical needle. In one preferred embodiment, the NR is comprised of an elastomeric material such as synthetic rubber. In a related embodiment, the NR is comprised of an elastomeric material with a hollow core wherein said NR is further comprised, on a sectional plane, of a first layer, an open space and a second layer wherein said NR affixes said surgical needle by a form lock. In another related embodiment, the NR is comprised of wire mesh. In yet another related embodiment, the NR comprises a soft plastic material.

In other embodiments, the cartridge is used in combination with a forceps comprising an NR which is comprised of a magnetic material.

In other embodiments, the cartridge is used in combination with a forceps comprising an NR comprised of an adhesive material.

In other embodiments, the cartridge is used in combination with a forceps comprising an NR which comprises an inducible gripping mechanism selected from a plunger and a clamp. Certain suturing procedures require the use of relatively large needles in order to pierce tough tissue, such as fascia and other tissues and to grasp the needle to pull the suture through the tissue. NRs suitable for grasping relatively large needles must exert sufficient pressure on the needle to maintain a grip on the needle while pulling the suture through the tough tissue. Thus, NRs suitable for grasping relatively large needles preferably comprise an inducible gripping mechanism such as a plunger mechanism or a clamp to receive and reversibly affix a large surgical needle. For example, the NR may be designed with an opening that allows a surgical needle to be inserted frictionlessly into the NR such that when the distal end of the arms are apart, a plunger mechanism which is dependent on the distance between the distal ends of the arms is activated and pushes on the part of the needle that is through the opening. The pushing force of the plunger acts to trap the needle and results in a grip on the needle thereby affixing the needle to the NR. Conversely, when the distal ends of the arms are in close proximity, the plunger mechanism responds by retracting the plunger, removing the pressure on the needle and the needle is released. Alternatively, the plunger mechanism may be independent of the distance between the distal ends of the forceps and may be activated manually by a surgeon, nurse or technician.

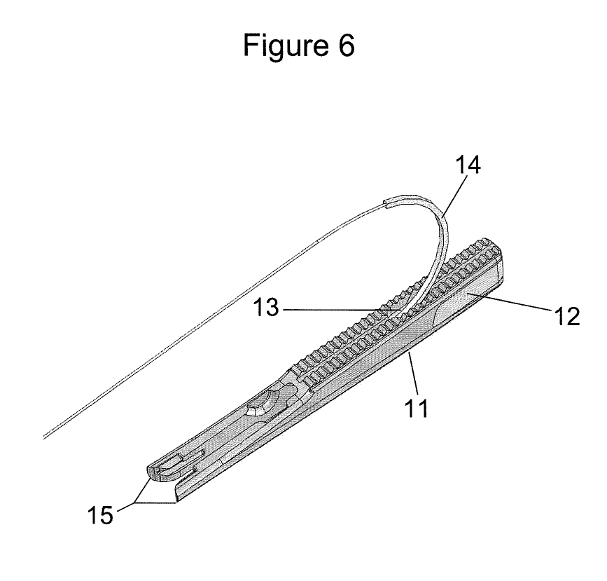

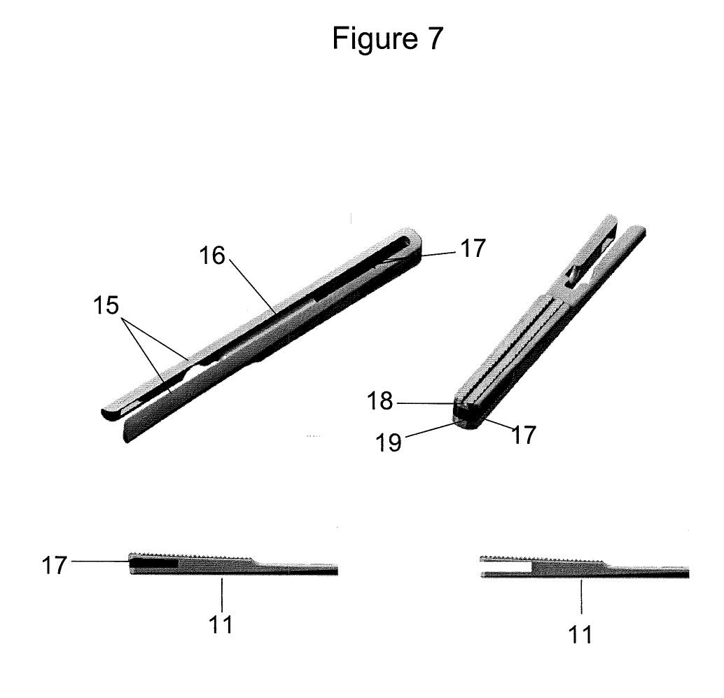

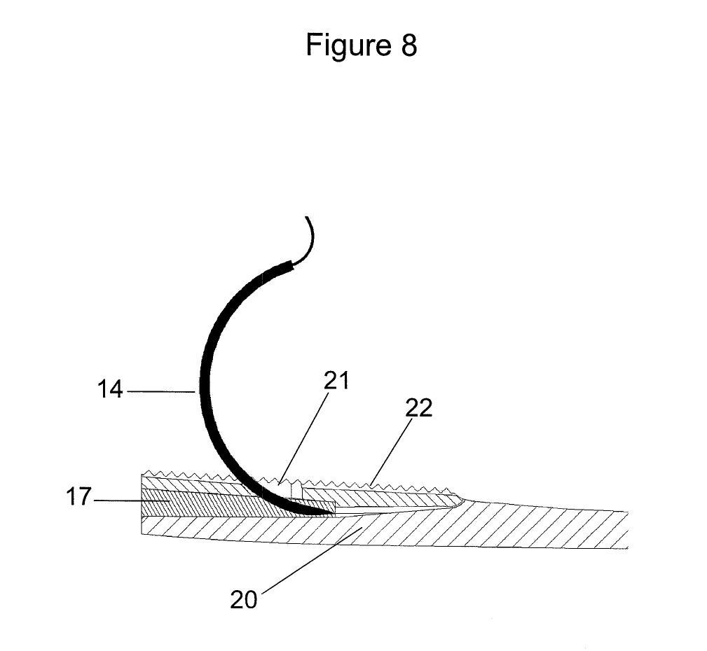

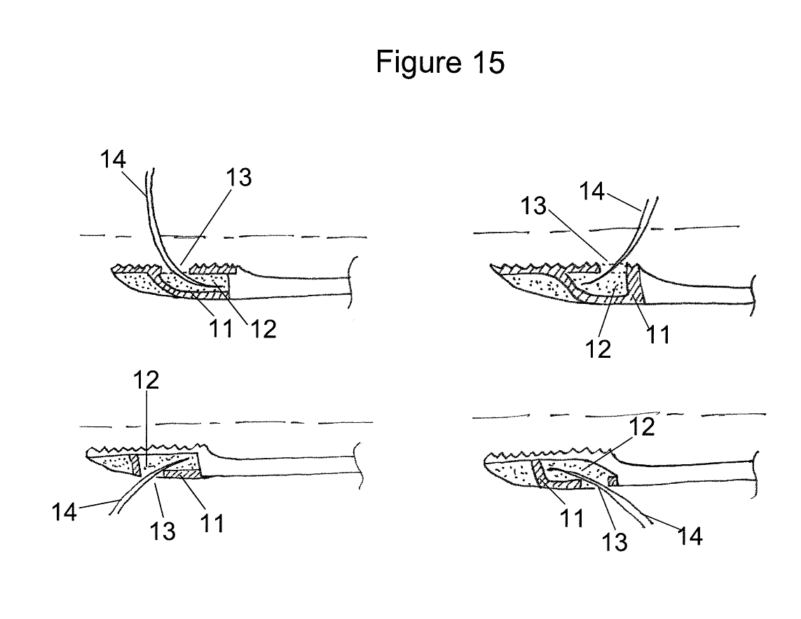

In other embodiments, the cartridge is used in combination with a forceps comprising an NR comprising a needle receiving and affixing layer (e.g. a soft plastic) at least partially surrounded by a needle-impenetrable layer (e.g. a hard plastic). The needle receiving and affixing layer is directly accessible to the surgical needle through a discontinuity in the needle-impenetrable layer. The NR may comprise, at a proximal portion, a means for reversibly attaching to the end of a forceps arm. The needle tip may be inserted so that the needle is parallel to the longitudinal axis of the NR or may be inserted at an angle relative to the longitudinal axis of the NR (i.e., slanted). Thus, the tip of the sharp needle will be covered during handling, disposal and possibly even during storage. Examples of this embodiment include the chamber NR illustrated in FIGS. 6 and 15 and the sandwich NR illustrated in FIGS. 7 and 8.

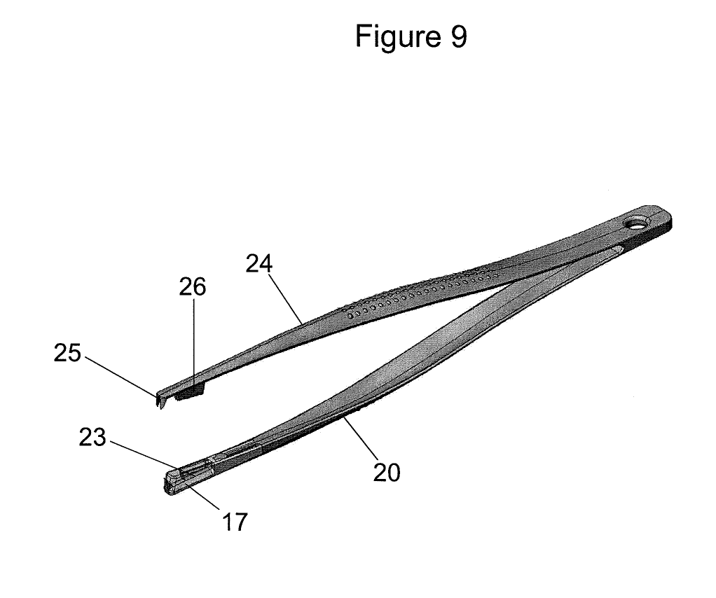

In still other embodiments, the cartridge is used in combination with a forceps comprising a first arm and a second arm, said first arm comprising an NR suitable for being pierced with a surgical needle at the distal end of the arm at an inner surface of the end of the arm, said NR further comprising a groove along the longitudinal axis of said forceps arm and said second forceps arm comprising a ridge at the distal end of the arm at an inside of the arm, wherein said ridge is positioned to fit into said groove when said forceps are in a closed position. When the forceps are in a closed position, the ridge exerts a force on a needle inserted into the NR at the distal end of the first forceps arm, trapping the needle against the inner surface of the first forceps arm, increasing the grip on the needle. The needle may be released by opening the forceps and pulling the needle in the reverse direction. Since the force on the needle is manually adjustable, this way of gripping the needle is inducible. The needle-affixing portion and/or the ridge portion of the NR may be disposable or may form a permanent part of the forceps. Examples of this embodiment are illustrated in FIGS. 9-11.

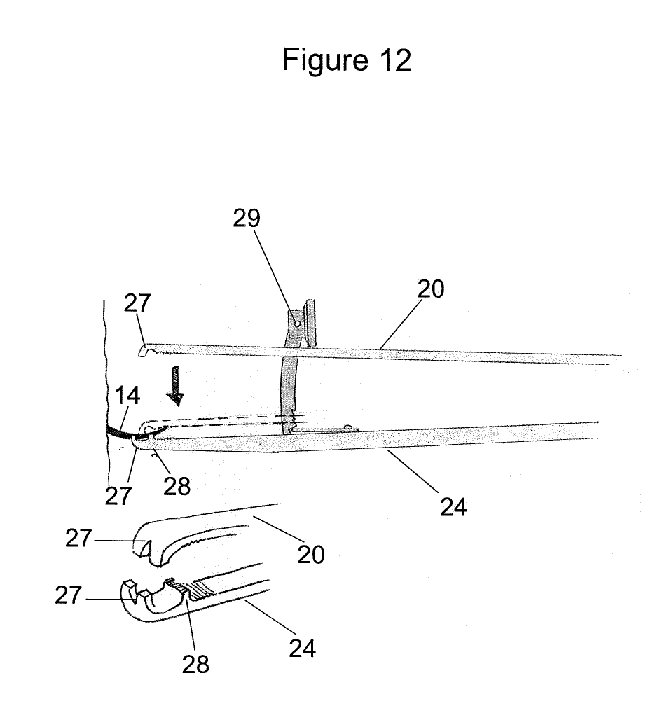

In another embodiment, the present invention provides a forceps suitable for gripping a large surgical needle comprising: (A) a first arm and a second arm connected at the proximal end; (B) a pair of prongs at the distal end of each forceps arm extending approximately perpendicular to the forceps longitudinal axis; (C) a first needle guiding means proximal to said pair of prongs on said second arm; and (D) optionally a second needle guiding means proximal to said first needle guiding means on said second arm, wherein the pair of prongs on said second arm opposes and is distal to the pair of prongs on said first arm when said forceps is in a closed position. The purpose of this device is to grab a large needle between the prongs of said first and second arms and hold it firmly without exposing the needle tip (and causing needle sticks). The locking means may consist of a catch which doesn't affect the forceps until the forceps is closed and squeezed firmly—this locks the forceps in a closed position. The forceps can either be disposable or reusable. Such an embodiment may be referred to as “fork-tip forceps” embodiment. An example of this embodiment is illustrated in FIG. 12.

In another embodiment, the present invention provides a method for preventing needle sticks or needle penetration accidents during a suturing procedure comprising the steps of (A) insertion of an arm of a forceps into a needle disposal and storage cartridge, said cartridge comprising a needle and a suture, thereby loading the forceps with an NR, a needle and a suture; and (B) removal of the said forceps arm from said cartridge; and (C) reinsertion of said forceps arm, after use, into said cartridge, thereby disposing of said NR, needle and suture; and (D) removal of said forceps arm from said cartridge. Preferably, the cartridge comprises a means for indicating the status of the cartridge (i.e., whether the cartridge is loaded with a fresh NR+needle+suture or whether the cartridge contains a used NR+needle+suture). Thus, instead of unpacking the suture, putting the needle manually in a needle holder, handing the needle/holder over to the surgeon, and getting the instruments back with a sharp unprotected contaminated needle for disposal, the method allows the suturing process to be performed without the need for direct human contact with the sharp surgical needle, thus reducing or preferably eliminating needle-stick injuries during surgery.

In another embodiment, the present invention provides a suturing system comprising: (A) at least one cartridge; (B) at least one NR; and optionally (C); a mounting mechanism such as a rail or other mounting surface for holding said cartridge(s); (D) a forceps; and (E) a clamp.

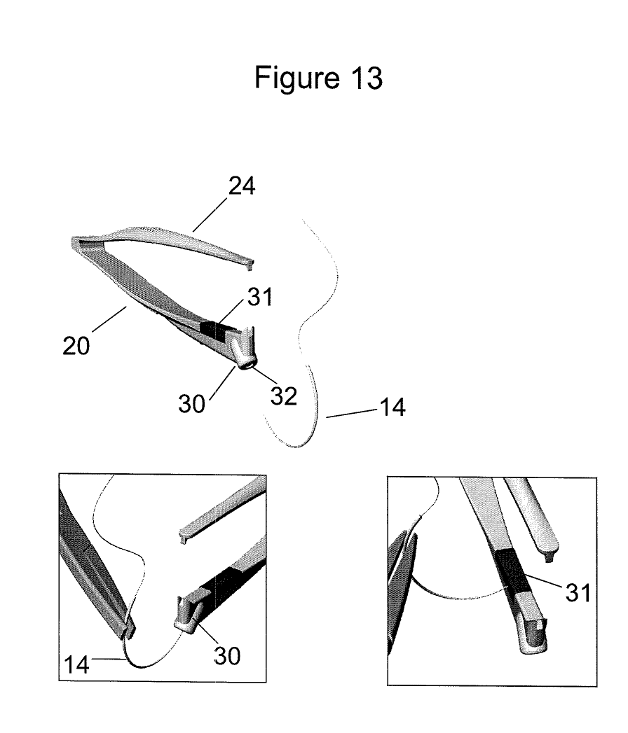

In yet another embodiment, the present invention provides a forceps suitable for gripping a large needle comprising a first arm and a second arm. A first NR is located at the distal end of at least one arm of the hybrid forceps and a second NR is located proximal to the first NR. The first NR is integrated into the at least one arm and comprises an opening at the distal tip of the arm through which a needle may be inserted and a needle affixing material which grips the needle after insertion. The purpose of the first NR is to hold the needle while the user makes a knot in the suture and to cover the needle tip during transfer of the forceps. The second NR may be any of the NR embodiments herein described. The hybrid forceps may be disposable or reusable. The second NR may be used during, e.g., a running suture. Such an embodiment may be referred to as a “hybrid forceps.” An illustration of this embodiment is provided at FIG. 13.

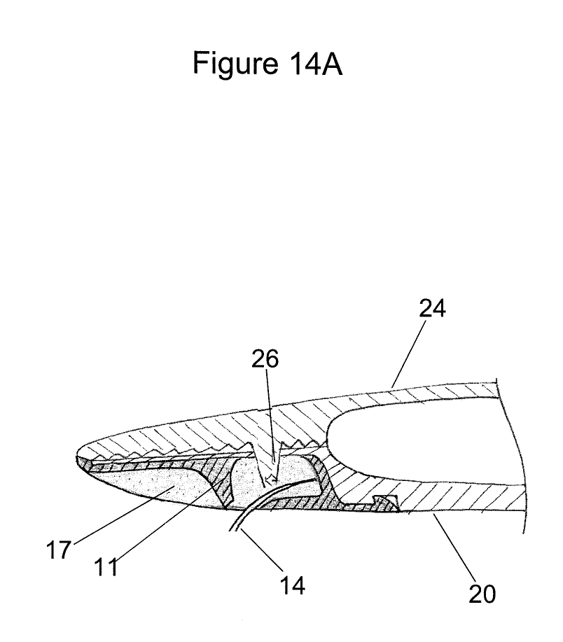

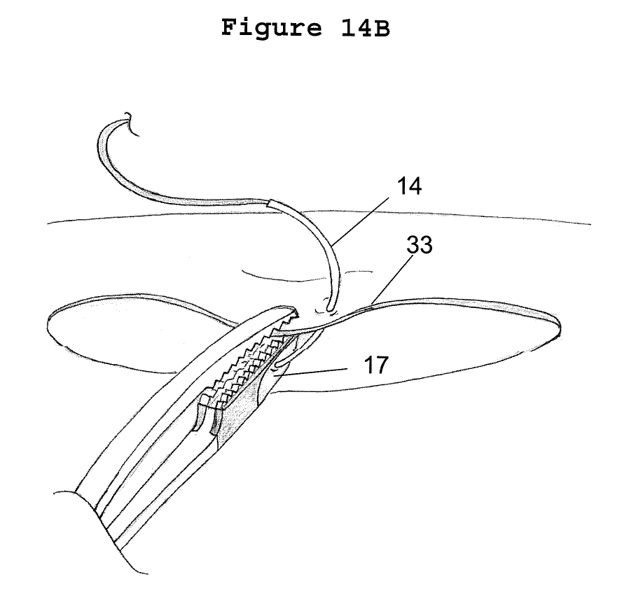

It will be understood that a forceps comprising an NR having a combination of features of any of the previously described NR embodiments may also be used in combination with cartridges of the invention and as part of suturing systems of the invention. Illustrations of forceps comprising an NR having features of both the NR ridge embodiment and the sandwich NR embodiment are provided in FIGS. 14A and 14B.

Identical reference numerals used in the figures refer to similar parts.

Referring first to FIG. 1, where reference numeral 1 indicates a needle storage and disposal cartridge according to the invention. The needle storage and disposal cartridge 1 comprises a NR 2 and a housing 3 comprising an opening 4 through which a forceps arm may be guided. FIG. 1 further shows that the disposal cartridge comprises a means for indicating to the user that the cartridge is “loaded” 5 with an NR and optionally a suture. The NR may be comprised of any needle receiving and retaining material suitable to be pierced with a surgical needle or may comprise an inducible gripping mechanism so long as the NR reversibly retains the needle until removed by the surgeon as discussed further below.

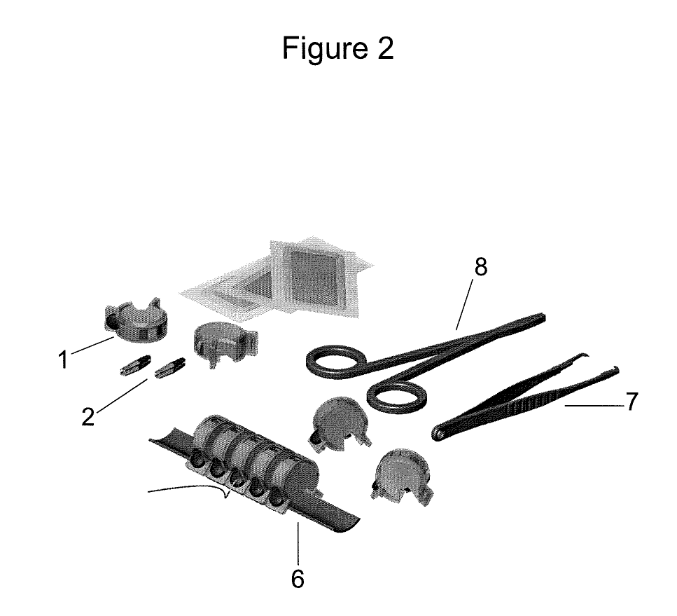

FIG. 2 shows a suturing system comprising a plurality of needle storage and disposal cartridges 1, a holding rail 6 for the cartridges, a suturing forceps 7, a needle holder 8 and NRs 2.

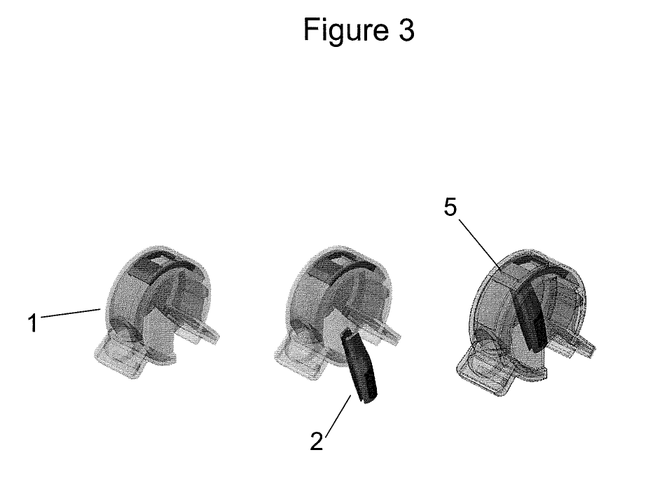

FIG. 3 demonstrates the loading of a cartridge 1 with a new NR 2. The means for indicating to the user that the cartridge is loaded 5 is provided by a color indicator which is green when the cartridge is loaded and red when the cartridge is unloaded.

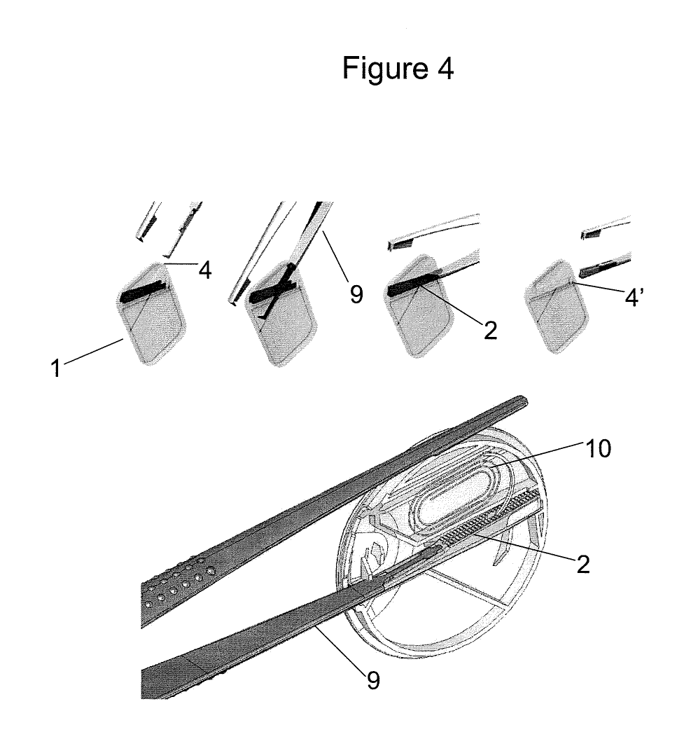

FIG. 4 (top panel) demonstrates a cartridge embodiment which enables the user to attach and detach the disposable NR to/from the reusable forceps. The cartridge consists of two parts which are connected, thus forming a small box. The user inserts the forceps arm 9 into a loaded cartridge 1 through a first opening 4 and then rotates the forceps clockwise until the forceps is approximately parallel to the cartridge, at which point the NR 2 is attached (i.e. snaps on) to the forceps arm 9. The user then pulls the forceps (plus NR) out of the cartridge through a second opening 4′ and the forceps is ready for use. Once surgery is over, the contaminated forceps with NR is inserted in the box in a second opening 4′ and rotated counter-clockwise. The NR remains horizontal to the cartridge (e.g. via snap fingers) and the forceps (without NR) may be removed from the cartridge via the first opening 4.

FIG. 4 (bottom panel) further demonstrates a cartridge 1 that is loaded with an NR 2 and a suture 10 affixed to the NR 2. A forceps arm 9 has been inserted into the cartridge and the NR 2 has been attached to the forceps arm 9.

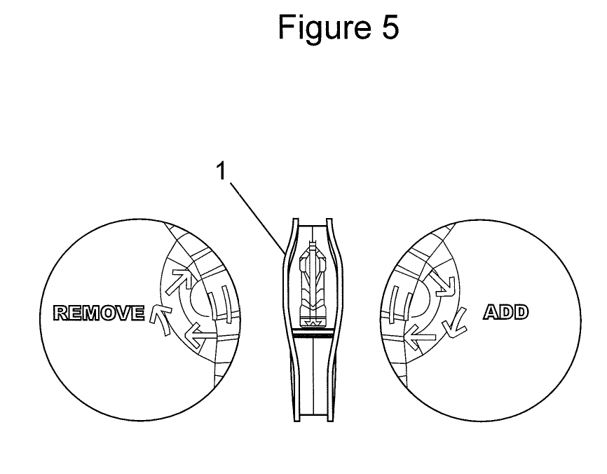

FIG. 5 demonstrates a cartridge 1 that is suitable for both the release of NR 2 and optionally suture 10 and disposal of NR 2 with suture 10. The opening 4 guides the NR 2 along with the needle affixed thereto, into the cartridge 1 for safe disposal.

FIG. 6 demonstrates a chamber NR embodiment. In this embodiment, the NR comprises a housing 11 preferably made of a hard plastic. The housing comprises a chamber 12 which is filled with a soft needle-affixing material (e.g. a soft plastic). The chamber 12 is accessible through a hole 13 in the housing 11. The tip of a suture needle 14 is inserted through the hole 13 in the housing 11 and penetrates the soft material in the chamber 12. The housing further comprises snap fingers 15 for attaching the NR to the distal end of a forceps arm. When attached to a forceps arm the hole 13 preferably faces the inner surface of the other forceps arm, although it is considered that the opening may face any direction. The tip of the sharp needle will be covered during handling, disposal and potentially even during storage.

FIG. 7 depicts a disposable sandwich NR embodiment. In this embodiment, the NR comprises a housing 11 which comprises a snap finger 15 extending along the longitudinal axis of the housing at the bottom 16 of the housing. The distal portion of the housing comprises a needle-affixing material 17 sandwiched between a top surface 18 and a bottom surface 19 of the housing. The distal portion of the housing is arranged to provide continuous access of the needle affixing material to a needle along the sides of the distal portion of the housing and at the distal tip of the housing and is also arranged so that the needle-affixing material 17 contacts a forceps arm when attached to a forceps. A needle is inserted into the needle-affixing material and is held in place due to the material specifications of the material and the fact that when the NR is attached to a forceps arm, the material is pressed between the forceps arm and an inner surface of the housing. Views of the bottom (top left panel), top (top right panel) and side (bottom panels) are shown. The side views depict the housing with (bottom left panel) and without (bottom right panel) the needle-affixing material.

FIG. 8 depicts a disposable sandwich NR embodiment (shown attached to a forceps arm 20) further comprising a hole 21 at the top 22 of the housing, located to allow a needle 40 inserted through the hole to pierce the needle-affixing material 17.

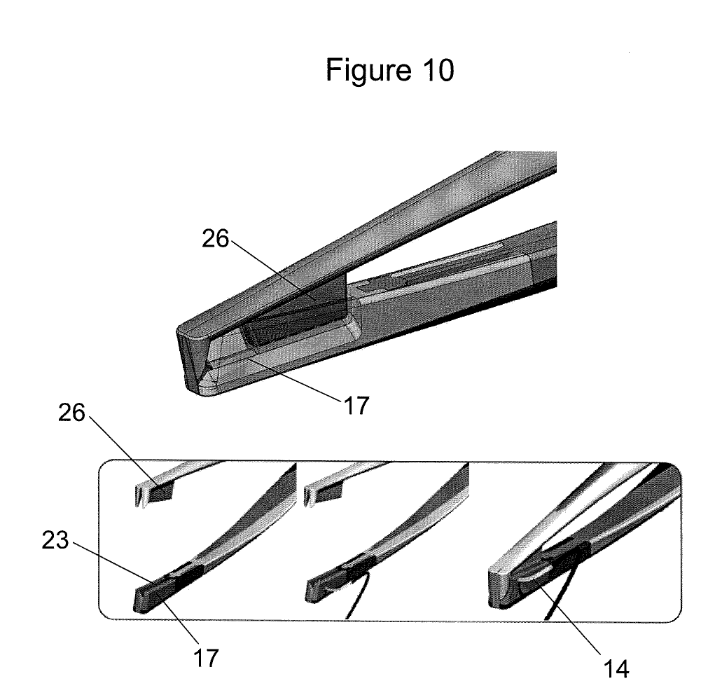

FIGS. 9 and 10 demonstrate a ridge NR embodiment. In this embodiment, a needle-affixing material 17 (e.g. soft plastic) is located at the distal end of a first forceps arm 20 at an inside of the arm. The needle affixing material 17 comprises a groove or slot 23 along the longitudinal axis of the forceps arm. In other words, the needle affixing material 17 locally consists of two thin walls. A second forceps arm 24 terminates at the distal end in a pair of prongs 25 approximately perpendicular to the longitudinal axis of the forceps arm and facing inward. Located proximal to the prongs at an inner distal surface of the second forceps arm 24 is a straight, flat ridge 26 (made of a rigid material such as metal or hard plastic) positioned to fit into the slot 23 of the first forceps arm 20 when the forceps are in a closed position. A needle 14 is inserted through both walls of the needle-affixing material 17 or through one wall and crossing the slot. The forceps are then closed, causing the ridge 26 to touch the needle 14 and press it toward the inner surface of the first forceps arm, thus increasing the grip on the needle. Since the force on the needle is manually adjustable this way of gripping the needle is inducible.

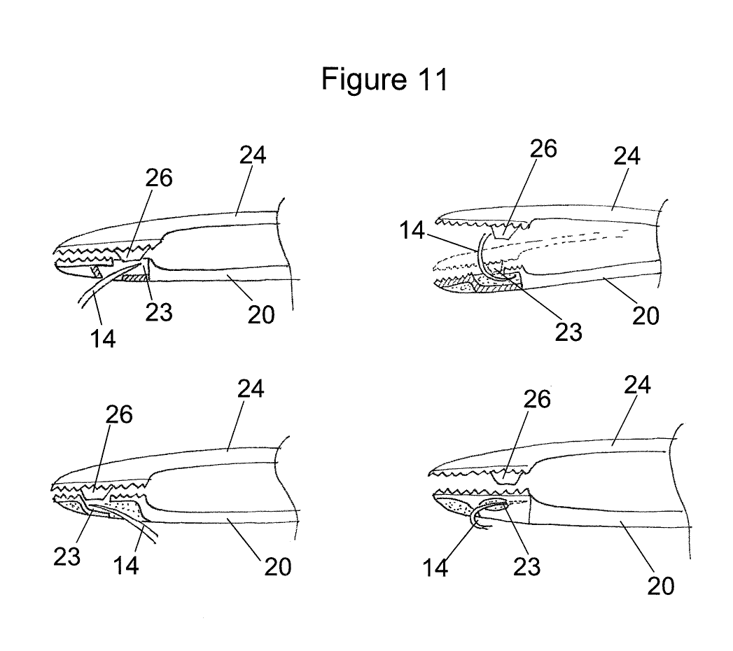

FIG. 11 illustrates side views of additional forceps embodiments. The illustrations demonstrate that the location of the ridge 26 on the inner surface of the second forceps arm 24 may vary. Correspondingly, the location of the slot 23 (made of a soft needle-affixing material) on the first arm 20 may vary. The illustrations also demonstrate that the location of needle insertion into the slot 23 on the first arm 20 may vary. For example, the needle 14 may be inserted into an outer surface of the first arm (upper left and lower left and right panels) or into an inner surface of the first arm (upper right panel). Alternately, the needle 14 may be inserted at the distal tip of the first arm. Regardless of the location of needle entry, in each embodiment the inserted needle rests within the slot on the first arm; when the forceps is closed, the slot is filled with the ridge thus pressing and grabbing the needle inserted into the slot. Importantly, in each embodiment (including embodiments where the needle insertion point is located at an outside of the forceps arm), the needle may be inserted into the forceps arm while the forceps grip the tissue. The soft needle-affixing material comprising the slot may be disposable and may comprise a means (e.g. snap finger) for reversibly attaching to a forceps arm. The forceps arms of each illustrated embodiment comprise a set of interlocking teeth on an inner distal surface (i.e., the tissue-gripping surface) of the arms.

FIG. 12 demonstrates the fork-tip surgical forceps suitable for gripping a large surgical needle and holding it firmly without exposing the needle tip. The forceps comprises a first arm 20 and a second arm 24, each of which has a V-shaped tip built of two prongs 27 (at the distal end) transverse to the arm and extending inward such that the prongs of the first arm are opposing the prongs of the second arm. The prongs 27 on the second arm 24 are distal to the prongs 27 on the first arm 20 such that when the forceps is closed the prongs 27 on the first arm 20 are adjacent to the prongs 27 on the second arm 24. The second arm 24 of the forceps further comprises a needle guiding means 28 for maintaining a surgical needle parallel to the forceps, located proximal to the tip. The means may comprise a grooved ridge or may comprise two prongs similar to those at the tip of the arm. A large needle 14 (e.g. barely sticking out of tissue) is placed between the prongs 27 on the first arm 20 and the prongs 27 on the second arm 24 and the forceps is firmly closed. The needle is pressed between the prongs 27 of each arm (like a wire in a wire cutter) and rests on the grooved ridge 28, maintaining the needle in a position parallel to the forceps. The forceps may further comprise a locking means 29 for maintaining the forceps arms in a fixed position. In one embodiment, the locking means consist of a catch. The catch doesn't affect the forceps until the forceps is closed and also squeezed firmly in position causing the forceps to snap reversibly into a fixed position. The forceps may be re-usable or may be disposable.

In a related embodiment, the second forceps arm comprises a second needle guiding means located proximal to the first needle guiding means. Preferably the second needle guiding means consists of a hollow metal shape or a shape filled with a flexible material into which the needle may be guided. This allows more of the needle tip to be inserted into the forceps while maintaining the needle in a position parallel to the forceps, minimizing the likelihood of needle sticks. The forceps can also be used as a regular forceps to grip tissue without damaging the tissue.

FIG. 13 depicts a hybrid forceps embodiment. The hybrid forceps comprises a first arm 20 and a second arm 24. A first NR 30 is located at the distal end of the first arm 20 of the hybrid forceps and a second NR 31 is located proximal to the first NR 30. The first NR 30 is integrated into the first arm 20 and comprises an opening 32 at the distal tip of the first arm through which a needle 14 may be inserted and a needle affixing material which grips the needle after insertion. The purpose of the first NR 30 is to hold the needle while the user makes a knot in the suture and to cover the needle tip during transfer of the forceps. The second NR 31 comprises a soft needle-affixing material forming a continuous surface with a portion of the first arm 20 but may be any of the NR embodiments herein described. The hybrid forceps may be disposable or reusable. The second NR may be used during, e.g., a running suture.

FIG. 14A illustrates a side view of a particular forceps embodiment. The forceps comprises an NR having features of both the NR ridge embodiment and the sandwich NR embodiment. A first forceps arm 20 comprises a housing 11 comprising a needle-affixing material 17, substantially as described in FIG. 7, at the distal end of the arm. However, the housing 11 further comprises a slot along the longitudinal axis of the forceps arm. A second forceps arm 24 comprises, at an inner distal surface, a ridge 26 as described in FIG. 10, positioned to fit into the slot of the first forceps arm 20 when the forceps are in a closed position. A needle 14 is inserted through the needle affixing material 17 and the forceps are closed, causing the ridge 26 to touch the needle 14 and press it toward the inner surface of the first forceps arm, thus increasing the grip on the needle.

FIG. 14B illustrates the forceps in a closed position gripping tissue 33. The needle 14 has been inserted into the needle affixing material 17 at a side of the NR/forceps. The figure demonstrates that the NR is suitable for receiving and affixing a surgical needle 14 while the forceps grip the tissue 33, despite the NR being located at the distal outside of the forceps arm and despite the location of the needle entry point on a side of the forceps arm.

FIG. 15 illustrates variations of the chamber NR. The illustrations demonstrate that the hole 13 leading to the soft needle-affixing material inside the chamber 12 may be located anywhere in the housing 11. The figure illustrates that, when attached to a forceps arm, the hole may be located at an inside surface of the forceps arm (top panels) or at an outside surface of the forceps arm (bottom panels). The illustrations also demonstrate that, irrespective of the location of the hole, the needle may be inserted with the needle pointing toward the proximal end of the forceps (left panels) or toward the distal end of the forceps (right panels).

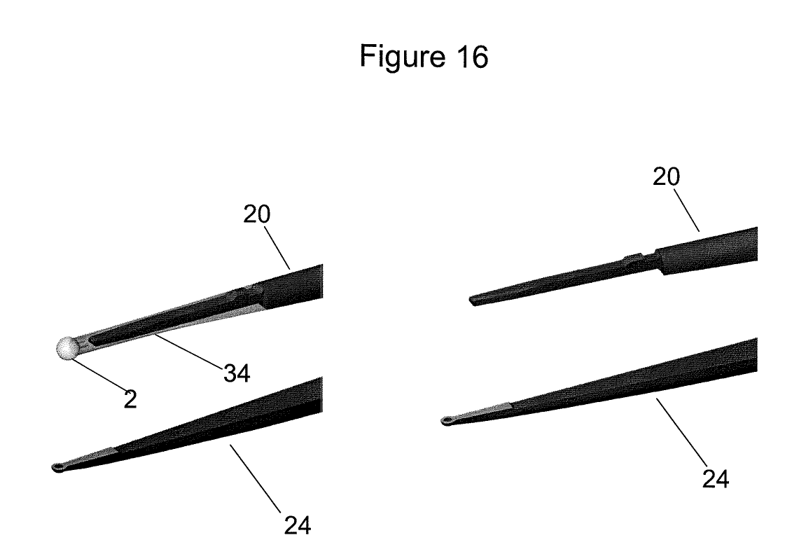

FIG. 16 illustrates a ‘micro’ forceps comprising an NR 2 suitable for use with relatively small surgical needles, such as those used in cardiovascular surgery. The NR 2 is provided on a holder 34 that is detachably placed on a first forceps arm 20. The NR is relatively small in size and is located at the distal tip of the holder. The first forceps arm 20 is shorter in length than the second forceps arm 24 and is designed such that when the holder 34 is positioned on the first forceps arm 20, the first forceps arm 20 including the holder 34 is substantially similar in length to the second forceps arm 24. Because the NR (with holder) is relatively small in size, cartridges for use with such ‘micro’ forceps may comprise several NRs (with holders).

Thus, the invention has been described by reference to certain embodiments discussed above. It will be recognized that these embodiments are susceptible to various modifications and alternative forms well known to those of skill in the art without departing from the spirit and scope of the invention. Accordingly, although specific embodiments have been described, these are examples only and are not limiting upon the scope of the invention.

What is claimed is:

1. A cartridge for storing and/or disposing of a needle receptor (NR) comprising an NR suitable for affixing a surgical needle, optionally a needle with or without suture, and a housing, said housing comprising at least one opening sized for insertion and removal of an arm of a forceps and arranged to allow affixation or removal of the NR, and optionally a needle with or without suture, to the distal end of said arm of a forceps upon insertion of said arm.

2. The cartridge of claim 1 comprising an indicator for identifying the status of said cartridge as either new or used.

3. The cartridge of claim 1 or claim 2 in combination with a forceps comprising a first arm and a second arm connected at a proximal end and defining a space between said first arm and said second arm which can be increased or decreased and wherein the needle receptor (NR) affixes at the distal end of an arm at an inside, outside and/or side of the end of an arm.

4. The cartridge of claim 3, wherein said NR is capable of being pierced with a surgical needle.

5. The cartridge of claim 4, wherein said NR is comprised of elastomeric material.

6. The cartridge of claim 5, wherein said elastomeric material is synthetic rubber.

7. The cartridge of claim 4, wherein said NR is comprised of an elastomeric material with a hollow core whereby said NR comprises, on a sectional plane, a first layer, an open space and a second layer such that said NR can affix said surgical needle by a form lock.

8. The cartridge of claim 4, wherein said NR is comprised of an elastomeric material with a core comprised of a filler material selected from a gel, a foam, a bead or a liquid, whereby said NR comprises, on a sectional plane, a first layer, said filler material, and a second layer such that said NR can affix said surgical needle by a form lock.

9. The cartridge of claim 4, wherein said NR comprises a soft plastic material.

10. The cartridge of claim 4, wherein said NR is comprised of wire mesh.

11. The cartridge of claim 1 or claim 2 in combination with a forceps comprising a first arm and a second arm connected at a proximal end and defining a space between said first arm and said second arm which can be increased or decreased, said first arm comprising a needle affixing material at the distal end of the arm at an inner surface of the end of the arm comprising a slot along the longitudinal axis of the forceps arm and said second forceps arm comprising a straight, flat ridge at the distal end of the arm at an inner surface of the end of the arm, wherein said ridge is positioned to fit into said slot when said forceps are in a closed position so as to exert a force on a needle.

12. The cartridge of claim 3, wherein said NR is comprised of magnetic material.

13. The cartridge of claim 3, wherein said NR is comprised of an adhesive material.

14. The cartridge of claim 3, wherein said NR comprises an inducible gripping mechanism selected from a plunger and a clamp.

15. The cartridge according to claim 1 or claim 2, wherein at least part of the cartridge is integrally formed with at least a part of the NR.

16. The cartridge according to claim 1 or claim 2 further comprising one or more additional NRs suitable for affixing a surgical needle.

17. A suturing system comprising: a plurality of cartridges according to claim 1 or claim 2 and optionally a rail or other holding mechanism for collectively holding said cartridges in the operating room.

18. The system of claim 17 further comprising a forceps and/or a needle holder arranged for attachment to the NR.

19. A method for preventing needle sticks during a suturing procedure comprising the steps of:
a. insertion of an arm of a forceps into a needle disposal and storage cartridge, said cartridge comprising an NR, a needle and a suture,
b. affixing the arm directly or indirectly to the NR, thereby loading the forceps with an NR, a needle and a suture;
c. removal of said forceps arm from said cartridge;
d. reinsertion of said forceps arm, after use, into said cartridge, thereby disposing of said NR, needle and suture; and
e. removal of said forceps arm from said cartridge.

20. The method of claim 19, further comprising retaining the NR within the cartridge to prevent subsequent reuse.

21. The method of claim 19 or claim 20, further comprising destroying at least part of the NR or needle.

22. The cartridge of claim 1 or claim 2 in combination with a hybrid forceps comprising a first arm and a second arm connected at a proximal end and defining a space between said first arm and said second arm which can be increased or decreased, wherein the NR comprises a first relatively weak NR at a distal end of an arm and a second relatively strong NR proximal to said first NR.

23. The cartridge of claim 1 or claim 2 in combination with a forceps comprising an NR at the distal tip of an arm at an inside, outside or side of the end of the arm, suitable for receiving and affixing a surgical needle while gripping the tissue.

24. The cartridge of claim 23 wherein said NR is a chamber NR.

25. The cartridge of claim 23 wherein said NR is a sandwich NR.

26. A forceps suitable for gripping a surgical needle comprising:
a. a first arm and a second arm connected at the proximal end;
b. a pair of prongs at the distal end of each arm extending approximately perpendicular to the forceps' longitudinal axis;
c. a first needle guiding means proximal to said pair of prongs on said second arm; and
d. optionally a second needle guiding means proximal to said first needle guiding means on said second arm;
wherein the pair of prongs on said second arm opposes and is distal to the pair of prongs on said first arm when said forceps is in a closed position.

27. The forceps of claim 26 wherein said first needle guiding means is a grooved ridge or a pair of prongs.

28. The forceps of claim 26 wherein said second needle guiding means comprises a hollow metal cylinder.

29. The forceps of claim 26 further comprising a means for reversibly locking said forceps in a fixed position.
